(19) United States
(12) Patent Application Publication
(10) Pub. No.: US 2009/0203002 A1
(43) Pub. Date: Aug. 13, 2009

(54) MESENCHYMAL STEM CELLS AS A VEHICLE FOR ION CHANNEL TRANSFER IN SYNCYTIAL STRUCTURES

(75) Inventor: Stephen Brown, South Burlington, VT (US)

Correspondence Address: KENYON & KENYON LLP, ONE BROADWAY, NEW YORK, NY, 10004, US

(73) Assignee: Columbia University, New York, NY (US)

(21) Appl. No.: 12/224,766

(22) PCT Filed: Mar. 6, 2007
(86) PCT No.: PCT/US2007/063366

§ 371 Date: Sep. 5, 2008

Related U.S. Application Data
(60) Provisional application No. 60/778,911, filed on Mar. 6, 2006.

(51) Int. Cl. C12Q 1/68; C12P 19/34; C40B 40/08

(52) U.S. Cl. 435/6; 435/91.2; 506/17

(57) ABSTRACT

The present invention provides a method of selectively amplifying fetal DNA sequences from a mixed, fetal-maternal source. This method utilizes differential methylation to allow for the selective amplification of trophoblast/fetal specific sequences from DNA mixtures that contain a high proportion of non-trophoblast/fetal DNA. The invention also provides methods of using the amplified fetal DNA sequences for aneuploidy detection.

### FIELD OF THE INVENTION

The present invention provides a method of selectively amplifying fetal DNA sequences from a mixed, fetal-maternal source. This method utilizes differential methylation to allow for the selective amplification of trophoblast/fetal specific sequences from DNA mixtures that contain a high proportion of non-trophoblast/fetal DNA. The invention also provides methods of using the amplified fetal DNA sequences for aneuploidy detection.

### BACKGROUND OF THE INVENTION

Large studies indicate that the incidence of whole-chromosome aneuploidy in newborns is between 1 and 2%. Hsu, In: A. M. (ed) Genetic Disorders and the Fetus. pp 179-248) (1998). Such chromosome abnormalities represent a significant cause of prenatal morbidity and mortality as well as a major cause of severe developmental delay in long-term survivors. Given the maternal age dependence of common trisomies and the marked rise in average maternal age, it is clear that the importance of screening aneuploidy will continue to increase. Reliable, inexpensive and non-invasive methods for the detection of aneuploidy during pregnancy are sorely needed.

Current options for aneuploidy testing are inadequate. At present, invasive testing by chorionic villus sampling (“CVS”) or amniocentesis is presented as an option to all women 35 years old and older and to other women with known elevated risk of aneuploidy. Thus, the majority of women, because they do not fall into these categories, are not offered invasive testing. Maternal age functions poorly as a screening test since most babies are born to women less than 35, and only about 1 in 250 women at age 35 will have a trisomy discovered by amniocentesis. Over the past 20 years, there have been major improvements in the efficiency of maternal serum screening for trisomy 21 (“T21”). The present state-of-the-art screening using maternal serum from two gestational time points as well as ultrasound has a ˜95% “sensitivity” for detection of T21 with a 5% false positive rate. See e.g. Wald N. J., et al. 111:521-31. (2004). There are, however, three major drawbacks with this type of testing. First, it does not provide a diagnosis, but rather a probability of Down syndrome. A “positive” result is defined as a risk of Down syndrome greater than or equal to a 35 year-old woman. Thus, most women with a “positive” result still have to consider that the chance of actually finding Down syndrome is still less than 1%. Second, this testing is limited to trisomy 21 and 18. The third problem is that it is only 95% sensitive. A 95% sensitivity in a screening test such as this has great value from the public health perspective, but for many patients, the 5% chance to miss T21 is unacceptable. Obviously non-invasive tests with much higher positive predictive values and higher sensitivities would be much more useful to patients and would immediately replace existing screening methods were they to become available.

Beginning about 12 years ago, the demonstration of fetal cells of various lineages in maternal blood caused great excitement. Techniques to purify such cells from maternal circulation were developed and the feasibility of prenatal diagnosis of a number of conditions was demonstrated. Nonetheless, such methods have not become practical. This is largely due to the paucity of fetal cells and the daunting problems of purifying them. Bianchi, D. W., et al., Br. J. Haematol. 105:574-83 (1999).

A large number of recent publications have documented that free fetal DNA is present in maternal plasma in virtually all pregnancies beginning early in the first trimester and continuing until delivery. Bischoff, F. Z., et al., Hum. Reprod. Update 11:59-67 (2005). Multiple studies have demonstrated that fetal sex can be determined by amplification of Y chromosome specific sequences in maternal plasma derived DNA and other reports have shown that fetal Rh blood group genotype can be determined as well. The absolute quantity of fetal DNA in maternal plasma is not large and depends on gestational age as well as recovery technique. Estimates, which are all based on quantitative PCR, suggest that there may be the equivalent of 50-200 genome equivalents of fetal DNA per ml of whole blood depending on gestational age and other parameters. Bischoff et al. 2005 Hum Reprod Update 11:59-67). The origins of maternal plasma derived DNA are unclear as well. Many investigators have assumed that it is likely to be derived from trophoblast, since this is the tissue most in contact with the maternal circulation. Direct evidence for this comes from a single publication, which identified placental mosaicism for a Y chromosome abnormality. Flori E, et al., Case report. Hum. Reprod. 19:723-4 (2004). Despite this early success in demonstrating the presence of fetal DNA in maternal plasma, the major problems in prenatal diagnosis, such as determining the presence of common trisomies has not been accomplished using maternal plasma derived DNA. This is due to the fact fetal DNA in maternal plasma exits as a mixture with maternal DNA, and the maternal component is generally more abundant. The ratio of fetal to maternal DNA seems to vary greatly from sample to sample and from method to method. At the minimum, it is about 1% of the DNA mass and at the maximum, could be much higher. Benachi A, et al., Clin. Chem. 51:242-4 (2005). Although PCR can be used to amplify very small amounts of DNA, there is no general method to selectively amplify fetal DNA. Any effort to amplify sequences common to the fetus and mother will only succeed in amplifying the maternal sequences. Thus far, it has only been through the use of primers specific for sequences that are not present in the maternal component (such as the Y chromosome) that selective amplification of fetal sequences has been accomplished. Physical separation techniques have been used to enhance the ratio of the fetal component of plasma derived DNA. Li Y, et al. Jama 293:843-9 (2005); Li Y, et al. Prenat. Diagn. 24:896-8 (2004a); Li Y, et al., Clin. Chem, 50:1002-11. Nevertheless, such techniques are unlikely to ever yield fetal DNA of sufficient purity to allow routine prenatal diagnosis.

Samples from the uterine cervix of pregnant women have been shown to contain fetal cells, and this represents another potential source of fetal DNA that could be used for noninvasive prenatal diagnosis. The literature on this topic has focused on two issues: 1) the reliability of recovering fetal cells from the uterine cervix and methods to improve it and 2) methods for separating fetal cells from the large background of maternal cells. Although various prenatal diagnoses have been performed using fetal cells and DNA derived from cervical samples, both of these issues remain significant hindrances to the routine use of this idea. The highest reported success rate for obtaining fetal cells from maternal cervical samples was 82%, and this was only when the semi-invasive technique of saline instillation was used. Cioni R, et al., Prenat. Diagn. 25:198-202 (2005). Both morphologic (Tutschek B, et al., Prenat. Diagn. 15:951-60 (1995); Bussani C, et al., Mol. Diagn. 8:259-63 (2004)) and immunologic (Katz-Jaffe M. G., et al., Bjog 112:595-600 (2004)) means have been used to separate fetal from maternal cells, and both have been shown to enrich for the percentage of fetal cells. However, DNA obtained from these methods is likely to be highly contaminated with maternal DNA. In addition, no large or systemic studies have been reported.

Clearly, a method that would allow the detection and analysis of trophoblast (and hence fetal) DNA sequences when they are in a mixture with maternal DNA would be extremely useful. Samples derived from either maternal plasma or from the uterine cervix could then be used directly for fetal analyses without extensive physical separation of maternal and fetal cells or DNA. Alternatively, physical methods for fetal DNA enrichment could be combined with trophoblast/fetal specific amplification to enhance the benefits of both. Thus, there remains a need for a method that would provide selective amplification of fetal DNA obtained from a mixed fetal/maternal DNA source. The present invention fulfills this need.

### SUMMARY OF THE INVENTION

The present invention provides a method for selective amplification of fetal DNA from a mixed fetal and maternal DNA sample comprising isolating DNA from a mixed fetal/maternal DNA sample; digesting the DNA with a methylation specific enzyme; ligating the digested DNA with a linker; subjecting the digested DNA to linker-mediated PCR amplification to obtain amplified PCR products; removing linker and primer DNA from the amplification products; circularizing the amplified PCR products; subjecting the circularized PCR products to exonuclease digestion to reduce any uncircularized DNA to single nucleotides; and subjecting the products to isothermal rolling circle amplification to selectively amplify fetal DNA to produce methylation-sensitive representations from fetal DNA.

Any methylation specific enzyme may be used and preferred enzymes HpyChIV-4, ClaI, AclI and BstBI. In preferred embodiments, the linker mediated PCR amplification is performed for 12 cycles. Further, in preferred embodiments, the exonuclease digestion is with Bal-31.

The present invention also provides a method of identifying a fetal-specific amplicon comprising, separately preparing methylation-sensitive representations from fetal DNA and whole-blood DNA using the method of selective amplification of fetal DNA described above; labeling the fetal DNA and the whole blood-DNA to produce labeled fetal DNA probes and labeled whole-blood DNA probes; hybridizing the labeled DNA probes to two identical arrays of oligonucleotides, wherein said arrays of nucleotides correspond to predicted restriction fragments for a given methylation-sensitive enzyme; and comparing the two arrays with each other to locate an oligonucleotide that hybridizes exclusively to a fetal DNA probe; and identifying the hybridized oligonucleotide from step d as a fetal-specific amplicon. In other embodiments, the fetal DNA probe and the whole-blood DNA probe are labeled with two different labels which allows the hybridization of labeled probes is to be performed on one array. The label may be a fluorochrome.

In preferred embodiments, the methylation sensitive enzyme used in the selective amplification is HpyCh4-IV.

Preferably the fetal DNA is obtained from first trimester pregnancies and more preferably from pregnancies of about 56-84 days.

The present invention also provides a library of fetal-specific amplicons produced by the method described above. The present invention also provides an array comprising the library of the fetal-specific amplicons.

The present invention also provides a method for determining whether the copy number for a predetermined locus of fetal DNA in a mixture of fetal and maternal DNA is either reduced or increased as compared to a normal copy number at the predetermined locus. The method comprises selectively amplifying the predetermined locus of fetal DNA in the test sample and in a control sample using the selective amplification of fetal DNA described above. The control sample has a normal copy number at the predetermined locus of fetal DNA. Next, the method comprises comparing the amount of the amplified DNA in the test sample to the amount of amplified DNA in the control sample; and correlating the reduced amount of amplified DNA to a reduced copy number or an increased amount of amplified DNA to an increase in copy number.

In another embodiment, the comparison includes normalization of the amplified DNA from the predetermined locus to DNA amplified from a control locus present at the same copy number in the test sample and the control sample.

The present invention also provides a method for determining in a test sample whether a copy number for a predetermined locus is either reduced or increased as compared to a normal copy number, comprising selectively amplifying fetal DNA in the test sample and in a control sample using the method of selective amplification of fetal DNA described above, wherein said control sample has a normal copy number at the predetermined locus; labeling DNA from the test sample and the DNA from the control sample from step a with a label to produce labeled test DNA probes and labeled control DNA probes; hybridizing labeled test DNA and labeled control DNA probes to an array of fetal-specific amplicons described above; comparing the amount of hybridization between the test DNA probes and the control DNA probes to determine signal strength; and correlating the signal strength with either an increase or decrease in copy number at the predetermined locus in the test sample.

In another embodiment, the test sample DNA and the control sample DNA are labeled with two different probes, which allows the hybridization to be performed on one array.

### BRIEF DESCRIPTION OF THE DRAWINGS

FIG. 1 depicts an ethidium stained agarose gel electrophoresis of blood and trophoblast/fetal DNA digested with several enzymes. “B” and “T” indicate blood and trophoblast/fetal samples, respectively. The horizontal white line indicates a molecular weight of about 1500 bp.

FIG. 2 provides representative examples of linker mediated amplifications. The top panel shows linker mediated PCR of DNA purified from four different samples of maternal serum. Products after 24 cycles are shown. The bottom panel shows linker mediated PCR of DNA purified from maternal serum. Products after 20 cycles are shown. Lanes 1 and 2 were collected without formaldehyde and lanes 3 and 4 were collected in tubes containing formaldehyde.

FIG. 3 depicts a gel showing amplified representation of AclI digested trophoblast/fetal and blood DNAs. The lanes are as follows: 1) marker; 2) trophoblast/fetal; and 3) blood. Lanes 4 and 5 are the same as 2 and 3 except no ligase was used during the linker ligation step. The white lines indicate the portion that was excised for cloning.

FIG. 4 shows the results of PCR using primers for specific AclI amplicons. PCR using primers for specific AclI was performed on 12 identically prepared representations of trophoblast/fetal and blood DNAs. Results for 4 primer sets are shown. A total of 10 such trophoblast/fetal “specific” primer sets were identified. “T” and “B” indicate that template was derived from trophoblast/fetal and blood respectively.

FIG. 5 shows PCR products using trophoblast/fetal specific primer sets on Bal-31 treated, isothermally amplified representations of trophoblast/fetal and blood DNAs. Each pair (“T” and “B”) are the results of one primer set on trophoblast/fetal and blood representation. The top panel shows visible products for all six trophoblast/fetal after 22 cycles of PCR no visible products for the blood samples. The bottom shows that after 35 cycles, primers 1 and 2 have visible products from blood representations.

FIG. 6 shows sequences of PCR products containing an informative SNP in trophoblast/fetal-specific amplicon. Panel A is from the input blood DNA. Panel B is form the input trophoblast/fetal DNA. Panel C is from a 20:1 mixture of the two input DNAs. Panel D is from the methylation-sensitive amplified representation of the mixed DNA sample. This shows that a heterozygous SNP present in the trophoblast/fetal DNA is amplified cleanly despite being present at only 5% and therefore undetectable in the starting mixture.

FIG. 7 shows PCR products of two starting DNAs as well as those amplified from the 20:1 mixtures. Primers that amplified a CA repeat polymorphism on a trophoblast/fetal-specific AclI amplicon were used to demonstrate selective amplification on a mixture of two DNAs. Panel A is input whole-blood DNA with genotype 198/202. Panel B is input trophoblast/fetal DNA with genotype 196/196. Panel C is 20:1 mixture with genotype 198/202. Panel D is methylation sensitive amplification of 20:1 mixture showing that that the trophoblast/fetal genotype is obtained despite 95% contamination with whole-blood DNA.

FIG. 8 shows data from a microarray described by Lucito et al. Genome Res 13:2291-305 (2003). Each point represents a log₁₀ mean ratio of intensity from 10,000 oligos spotted on a glass array and comparatively hybridized. All addresses representing BglII fragments with internal HindIII sites are to the far left. Mean ratios for fragments with internal HindIII sites are generally well above 1:1 (10⁰).

### DETAILED DESCRIPTION OF THE INVENTION

The present invention provides a method for specific amplification of fetal DNA sequences from a mixed, fetal-maternal source. Generally the method involves the steps of: isolating DNA from a mixed fetal-maternal source; subjecting the isolated DNA to linker-mediated PCR; circularization of the amplified PCR products; exonuclease digestion; and finally isothermal rolling circle amplification.

The DNA may be obtained from a mixed fetal-maternal source of DNA.

Fetal-Maternal Source of DNA

Invasive procedures such as chorionic villus sampling (“CVS”) and amniocentesis can provide pure fetal DNA that can be used for prenatal diagnosis. Although these procedures are routinely used, they have associated risks. On the other hand, several non-invasive routes for obtaining fetal DNA exist: recovery of cell free DNA that is present in maternal plasma and through the recovery of exfoliated fetal cells from the maternal uterine cervix. Nevertheless, efforts to use fetal DNA for routine prenatal diagnosis have been constrained by the fact that the fetal DNA exists in an admixture with maternal DNA.

The methods of the present invention enable the use of fetal-maternal DNA mixtures as it utilizes the differences in DNA methylation of fetal and maternal DNA to provide amplification of fetal-specific sequences from mixed fetal/maternal DNA samples. By taking advantage of these methylation differences, the present invention provides a method of selective amplification of fetal sequences from an admixture of fetal and maternal DNA. This method thus opens up the possibility of performing prenatal tests for such things as common chromosomal abnormalities on DNA derived from maternal plasma or from a cervical swab.

As discussed above, the present invention relies on the difference of methylation between fetal and maternal DNA.

### Differences in Methylation of Fetal and Maternal DNA-Hypomethylation of Trophoblast/Fetal DNA

DNA methylation is an epigenetic event that affects cell function by altering gene expression and refers to the covalent addition of a methyl group, catalyzed by DNA methyltransferase (DNMT), to the 5-carbon of cytosine in a CpG dinucleotide. Methods for DNA methylation analysis can be divided roughly into two types: global and gene-specific methylation analysis. The methylation state of mammalian DNA undergoes dramatic changes during fetal development. It is thought that at the time of conception both maternal and paternal genomes are extensively methylated. In the course of the first few cell divisions, this methylation is largely “erased” and then later, by the time of implantation, de-novo methylation occurs and a large amount of methylation is present again. Bird A, Genes. Dev. 16:6-21 (2002). In all adult tissues that have been studied, a high percentage (up to 85%) of CpG dinucleotides are methylated. Gruenbaum Y, et al., FEBS Lett 124:67-71 (1981).

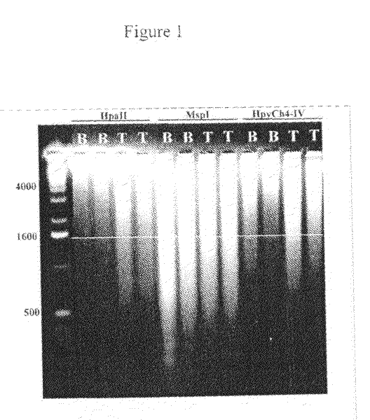

Knowledge of which sequences are methylated is currently rudimentary and is largely based on studies performed in the 1980s that relied on simple techniques such as comparisons of methylation and non-methylation sensitive digestions of DNA. Bird AP (1980) Nucleic Acids Res. 8:1499-504 (1980). There is a great deal of current interest in methylation and its role in regulation of gene expression. All existing literature on methylation of genomic DNA is based on samples derived from fetal or adult sources such as liver and whole-blood. To date, there have been no systematic studies of methylation in extra-embryonic tissues such as trophoblast/fetal. In the course of performing prenatal diagnosis for disorders such as Prader-Willi Syndrome and Fragile X Syndrome, it has been noted that trophoblast/fetal DNA is relatively hypomethylated in comparison with DNA derived from blood, liver or skin. Iida T., Hum. Reprod. 9:1471-3 (1994). This difference is most apparent when performing Southern blots that utilize methylation sensitive restriction enzymes. When most mammalian DNA is digested with a methylation sensitive restriction enzyme with a four base recognition sequence (e.g. HpaII), it is striking to see that the majority of the DNA remains high molecular weight. The average molecular weight of fragments is above 15 kb whereas the predicted frequency of HpaII predicts a much smaller average fragment size. By looking at such digests (See FIG. 1), one could guess that at least 80% of HpaII sites do not cut. Although it cannot be appreciated in the figure, if one runs gels with a higher percentage of agarose, it is clear that there is bimodal distribution of fragments, with one group being quite small and the other quite large. These observations basically recapitulate the discovery of so called “HpaII Tiny Fragment” or “HTF islands” reported in 1983. Cooper D. N., et al., Nucleic Acids Res. 11:647-58 (1983). If one digests the same DNA with MspI (same recognition sequence as HpaII but not methylation sensitive), the average molecular weight of fragments is much closer to the predicted size. However, the same experiment using DNA prepared from first trimester trophoblast/fetal yields quite different results. There is an obvious decrease in average molecular weight of HpaII digested trophoblast/fetal DNA, although it is still not equal to that obtained by MspI digestion. This clearly demonstrates that DNA prepared from trophoblast/fetal is relatively hypomethylated (less methylated), and it makes the important prediction that the hypomethylated areas are much more broadly distributed throughout the genome than are CpG or “HTF” islands.

It is difficult to precisely determine the degree of hypomethylation in trophoblast/fetal DNA relative to whole-blood DNA, but densitometry performed on digests of trophoblast/fetal vs. whole-blood DNA using the enzyme HpyChIV-4 (similar to that in FIG. 1) indicate that for a fragment window of 500-1,000 bp, the density of the resulting smear is consistently 2-3 fold higher for trophoblast/fetal samples. This implies that in this size range, HpyCh4-IV digested trophoblast/fetal DNA contains 2 to 3 times more fragments than whole-blood DNA.

The gestational age dependence of methylation differences between trophoblast/fetal and whole-blood derived DNA has not yet been fully investigated. In a series of 10 samples ranging in gestational age from 9 to 20 weeks, no differences in digestions performed with HpaII and HpyCH4-IV were detected. However, experience with methylation sensitive Southern blot analysis of the Prader-Willi and Fragile X loci indicates that by mid second trimester, there may be more methylation of trophoblast/fetal DNA than is present in the first trimester. Thus preferably the mixed DNA samples are obtained from pregnancies of 10-13 weeks (by LMP).

A method of the present invention thus provides for selective amplification of fetal DNA from a mixed fetal and maternal DNA sample utilizing the methylation differences between fetal DNA and maternal DNA discussed above. As noted previously, generally the method involves the steps of: isolating DNA from a mixed fetal-maternal source; subjecting the isolated DNA to linker-mediated PCR; circularization of the amplified PCR products; exonuclease digestion; and finally isothermal rolling circle amplification.

Methods of the present invention comprise subjecting the isolated DNA to linker-mediated PCR.

Linker-Mediated PCR

Generally, linker-mediated PCR begins with digesting DNA with a restriction enzyme and ligating double stranded linkers to the digested ends. PCR is then performed with a primer that corresponds to the linker and fragments up to about 1.5 kb are amplified. See Saunders, R. D., et al., Nucleic Acids Res. 17:9027-37 (1989) and Lisitsyn, N. A., et al., Cold Spring Harb. Symp. Quant. Biol. 59:585-7 (1994). Using this technique, it has been possible to amplify DNA from a single cell and to subsequently detect aneuploidy by using the amplified product to perform comparative hybridization. Klein, C. A., et al., Proc. Natl. Acad. Sci. USA 96:4494-9 (1999). In another study, amplified representations were used to detect single genomic copy number variations by using them as hybridization probes to BAC microarrays. Guillaud-Bataille, M., et al., Nucleic Acids Res. 32:e 112 (2004).

In this method, the frequency of digestion of the restriction enzyme determines the complexity of the amplified product that results. By choosing an enzyme that cuts infrequently, the complexity of the amplified representation can be reduced to a fraction of the starting genomic DNA making the subsequent hybridization step much easier to perform. This has been particularly useful in settings where one wishes to perform comparative hybridizations between two complex genomic sources. A striking example is a technique called “ROMA” (Representational Oligonucleotide Microarray Analysis) that has been instrumental in revealing a high degree of genomic copy number variation in humans. Lucito, R., et al., Genome Res. 13:2291-305 (2003); Sebat, J., et al., Science 305:525-8 (2004); Jobanputra, V., et al., Genet Med 7:111-8 (2005).

Example 1 shows successful use of linker-mediated amplification of DNA isolated from plasma of pregnant women. Before amplification, the CpG methylation sensitive enzyme HpyCh4-IV was used to digest purified DNA. After digestion, linkers were annealed and ligated to the digested DNA and finally PCR was performed using the top strand of the linker pair following a published protocol. See and Example 1 and Guillaud-Bataille, M., et al., Nucleic Acids Res. 32:e112 (2004). Notably, it was determined that maternal blood collection methods should preferably not involve formaldehyde.

Example 2 shows successful linker-mediated methylation specific amplification of trophoblast/fetal DNA. Trophoblast/fetal DNA as well as DNA samples from whole blood were digested with the CpG methylation sensitive enzyme AclI. Similar to example 1, after enzyme digestion, linkers were annealed and ligated to the digested DNA. Finally PCR was performed using the top strand of the linker pair following the same PCR protocol set forth in Example 1. Notably, trophoblast/fetal DNA consistently yielded more robust and differently appearing PCR products than did whole blood. However, it was determined that despite the fact that a CpG methylation sensitive enzyme was used, non-trophoblast/fetal DNA (i.e. DNA from whole blood) was still amplified. Accordingly, the present inventors determined that linker-mediated PCR amplification alone was not adequate for a specifically amplifying trophoblast/fetal DNA.

Accordingly, in the linker-mediated PCR step of the present invention, a mixed sample of DNA is obtained and digested with a CpG methylation sensitive enzyme to form digested DNA with digested ends. Methylation sensitive enzymes are known in the art and include, but are not limited to, HpyChIV-4, ClaI, AclI, and BstBI.

By using a CpG methylation sensitive restriction enzyme to cleave DNA prior to linker ligation, only fragments defined by unmethylated sites can be amplified. In a setting in which there is a mixture of DNAs from two different sources, one less methylated than the other, digestion with a methylation sensitive enzyme followed by linker ligation and amplification allows the selective amplification of fragments defined by differentially methylated sites. This idea has been used in conjunction with “representational difference analysis” to probe methylation differences between normal and cancerous tissues. See Ushijima, T., et al., Proc Natl. Acad. Sci. USA 94:2284-9 (1997) and Kaneda, A., et al., Acad. Sci. 983:131-41 (2003). The degree to which differential amplification can be achieved by this approach depends (in part) on the degree to which the methylation differences are present. For instance, if a given site is 100% methylated in one DNA and 0% methylated in another, then a high degree of differential amplification is expected.

Little is currently known about the degree to which many genomic sites are methylated. The tools for determining methylation state, namely Southern blot and bisulfite sequencing, have generally shown that specific sites are either completely methylated or completely unmethylated, suggesting that the methylation state of given sites are very tightly regulated and maintained. This idea is further corroborated by the fact that the two alleles of certain loci are precisely differentially methylated in regions of the genome that exhibit imprinting and dosage compensation. However, the number of specific sites that have been extensively investigated is limited. Also, the detection methods (Southern blot and bisulfite sequencing) are not able to distinguish between subtle differences in degree of methylation. However, using methods of the present invention, however, it was determined that there is a highly specific differential amplification of trophoblast/fetal sequence.

As noted above, methylation of mammalian genomes is highly non-random. GC rich regions and CpG or “HTF” islands are relatively hypomethylated while AT rich sequence is relatively more methylated. For example, more than 90% of sites for the rare-cutting GC rich enzyme, NotI, are located in hypomethylated, GpG islands resulting in much more frequent digestions with this enzyme than might be naively predicted. See Fazzari, M. J., Greally J M, Nat. Rev. Genet. 5:446-55. (2004). Since the present invention utilizes methylation differences to differentially amplify trophoblast/fetal specific sequences, and since it seems very likely that CpG islands are hypomethylated in both trophoblast/fetal and other DNAs, the methods focus on CpG methylation in non GC rich DNA. To this end, restriction enzymes that contain a methylation sensitive CpG, but otherwise consist of AT are preferred. Four enzymes fall into this category. One is a four base enzyme, HpyChIV-4, and cuts at ACGT. The remaining three enzymes are six base enzymes: ClaI, AclI and BstBI with sequences ATCGTA, AACGTT and TTCGAA respectively.

Informal analysis of 10 million bases of randomly chosen genomic indicated that sites for these enzymes are almost never present in CpG islands. Restriction maps of NotI sites were compared to those of AclI, ClaI and BstBI. Analysis showed that these AT rich sites are not clustered at CpG islands and on the contrary, they essentially never occur within CpG islands.

Surprisingly, recognition sites for AclI, BstBI and ClaI are also quite rare. It appears that the human genome contains only ˜150,000 AclI sites instead of the 750,000 that would be predicted under the assumption that genomic sequence is balanced with respect to the frequency of A, C, T and G. This ˜80% reduction in the number of actual compared to expected AclI sites is due to the relative paucity of CpG dinucleotides. Because linker mediated PCR can only amplify fragments up to about 1,500 bp in length, we searched for all predicted AclI fragments between 400 and 1500 bp and found that the total number in the human genome is only ˜15,000. If one assumes that up to 90% of predicted AclI sites in whole-blood DNA are blocked by methylation (a conservative assumption given the fact that CpG methylation is increased in AT rich sequence), the true number of expected fragments in this size range might be as 1,000-2,000. In aggregate, these ˜2,000 fragments would represent less than 0.1% of all genomic sequence. The same calculation for trophoblast/fetal DNA (assuming that only ˜80% of sites are methylated) predicts about 2,000-4,000 amplifiable AclI fragments. This calculation makes the important prediction that about half of all amplified fragments in a methylation-sensitive representation of trophoblast/fetal DNA would be expected to be “specific” to or highly enriched compared to a similarly prepared whole-blood representation.

After the DNA obtained from the mixed sample is digested with a methylation specific enzyme as discussed above, the DNA is then ligated to a linker. Preferably the linker has a built in restriction site, which will later be used to provide compatible sticky ends necessary for the circularization step. Any restriction enzyme site that produces sticky ends upon digestion may be used. For example, MluI provides sticky ends. After ligating the linker, the resulting DNA is amplified using a primer that binds to a site within the linker. PCR amplification is then carried out. The number of cycles may vary but preferably the number of cycles will create a size-selected representation of digested fragments. In preferred embodiments 5 to 15 cycles of amplification are carried out. In a more preferred embodiments 8-14 cycles of amplification are carried out. In a most preferred embodiment, 12 cycles of amplification are carried out

In addition to linker-mediated PCR amplification, the methods of the present invention further comprise circularization of the amplified PCR products; exonuclease digestion; and finally isothermal rolling circle amplification (discussed below), as the present inventors determined that linker-mediated PCR was not sufficient to specifically amplify fetal DNA. Example 2 shows that some non-fetal DNA sequences were amplified.

### Circularization of Amplified PCR Products

After the cycles of amplification are carried out, the amplified products are then digested with an enzyme that cleaves off the linker. For example, if the linker had a MluI site built into it, then the products would be subjected to a MluI enzyme digest. Following digestion to cleave the linker, low molecular weight DNA (linker and primer DNA) is removed. Any suitable method to remove low molecular weight DNA may be used, such as agarose gel purification or column purification. In preferred embodiments, column purification is used.

The purified DNA is then diluted to create a very dilute solution. This DNA is then treated with T4 DNA ligase overnight to allow circularization by allowing ligation of the sticky ends created by the earlier enzyme digest. By digesting and ligating in a very dilute solution (e.g. 0.5 ml in 1× ligation buffer), intra-molecular self-ligation (circularization) of molecules with compatible sticky ends is strongly favored. The original starting DNA that has been melted and partially re-annealed 12 times (during the PCR amplification) is very inefficiently digested and circularized. Further, the non-specifically amplified products that lack appropriate ends will also be highly unlikely to form covalently closed circles.

### Exonuclease Digestion

After the DNA is precipitated (using methods commonly known in the art) and resuspended in a suitable buffer such as water, the ligation mixture is treated by extensive digestion with an exonuclease that attacks the ends of single stranded and double stranded DNA (e.g. nuclease Bal-31). The circular molecules created by ligation are resistant to digestion, but extensive digestion will reduce any linear molecules to single nucleotides. This digestion is used to thus eliminate the starting genomic DNA as well as non-specifically amplified products. Alternatively, instead of a single exonuclease such as Bal-31, a mixture of exonucleases could be used. For example, one enzyme attacks single stranded DNA (mung bean exonuclease) and the other enzyme attacks double stranded DNA (Lamba exonuclease) and wherein neither of the enzymes have endonuclease activity and neither cleaves double stranded DNA at nicks.

By the term extensive digestion, it is meant that a sufficient amount of enzyme is used so as not to be limiting and that the time allowed for digestion is long enough not to be limiting. For example, in one embodiment 2 units of Bal-31 nuclease is used in the digestion mixture and allowed to proceed for 45 minutes. The units are defined functionally as the amount of enzyme needed to digest 400 bases of linear DNA in a 40 ng/ul solution in 10 minutes.

### Isothermal Rolling Circle Amplification

The nuclease treated ligations are then used as template for isothermal rolling circle amplification. Isothermal rolling circle amplification is known in the art and is generally a one cycle amplification of circular DNA using exonuclease-resistant random primers and a DNA polymerase with great processivity. Any isothermal rolling circle amplification procedure may be used. A commonly known kit if available from Amersham and is used following the manufacturer's recommendations.

Using a method of the present invention, the inventors were able to demonstrate specific amplification of the trophoblast/fetal component (hence fetal DNA) of mixed DNA samples to produce methylation-sensitive representations from fetal DNA. See Example 4.

The present invention also provides a method of identifying a fetal-specific amplicon. See example 5 for a detailed explanation. This method comprises separately preparing methylation-sensitive representations from fetal DNA and whole-blood DNA using the method of selective fetal DNA amplification described above. Fetal-specific amplicon means an amplicon that will amplify from trophoblast/fetal DNA but not other DNA using the methods described herein. Trophoblast/fetal DNA is DNA that is hypomethylated as compared to adult DNA. Restriction enzymes that are sensitive to methylation will cleave hypomethylated fetal loci and will not cleave methylated maternal loci.

The methylation-sensitive representations from fetal DNA are labeled with a first fluorochrome and the whole blood-DNA is labeled with a second fluorochrome different from first fluorochrome to produce labeled fetal DNA probes and labeled whole-blood DNA probes. The labeled probes are allowed to hybridize with an array of oligonucleotides corresponding to predicted restriction fragments for a given methylation-sensitive enzyme. Alternatively, if two separate identical arrays are used, the probes need not be labeled with different fluorochromes. The array(s) are studied to locate oligonucleotide(s) that hybridize exclusively to a fetal DNA probe. These oligonucleotides are identified as a fetal-specific amplicon.

In preferred embodiments, the methylation sensitive enzyme used in the fetal specific DNA amplification is HpyCh4-IV.

Preferably the fetal DNA is obtained from first trimester pregnancies of about 56-84 days since it is suspected that differences in fetal DA and maternal DNA methylation are more pronounced in early gestation.

The present invention also provides a fetal-specific amplicon produced by the method described above. The present invention also provides an array comprising a library of the fetal-specific amplicons identified using the methods of the present invention.

The present invention also provides a method for determining whether the copy number for a predetermined locus of fetal DNA in a mixture of fetal and maternal DNA is either reduced or increased as compared to a normal copy number at the predetermined locus. See example 6 for a detailed discussion. This method comprises selectively amplifying the predetermined locus of fetal DNA in the test sample and in a control sample using the method of selective fetal DNA amplification discussed above. The control sample has a normal copy number at the predetermined locus of fetal DNA.

The relative amount of the amplified DNA for a given locus in the test sample is compared to the relative amount of amplified DNA for the same locus in the control sample. A reduced amount of amplified DNA is correlated to a reduced copy number and an increased amount of DNA is correlated to an increase in copy number.

In a preferred embodiment, the comparison includes normalization of the amplified DNA from the predetermined locus to DNA amplified from a control locus present at the same copy number in the test sample and the control sample.

The present invention also provides another method for determining in a test sample whether a copy number for a predetermined locus is either reduced or increased as compared to a normal copy number. See Example 7 for a detailed discussion. This method comprises selectively amplifying fetal DNA in the test sample and in a control sample using the method of selective fetal DNA amplification discussed above. The control sample has a normal copy number at the predetermined locus.

The DNA from the test sample and control sample from step a is labeled to provide labeled probes. The labeling is performed to provide a means of detecting hybridization. For example if one array will be used, the DNA from the test sample is labeled with a first fluorochrome and the DNA from the control sample is labeled with a second different fluorochrome. Alternatively, if two separate identical arrays are used, one for the test DNA probes and one for the test sample DNA probes, two different labels are not necessary.

After labeling the DNA probes, they are hybridized to an array of fetal-specific amplicons as described and produce by the methods of the claimed invention. The amount of hybridization between the test DNA probes and the control DNA probes is measured to determine signal strength. A strong signal from the test DNA as compared to the control DNA correlates with an increase in copy number. A weak signal from the test DNA as compared to the control DNA correlates with a decrease in copy number.

### EXAMPLES

### Example 1

### Linker-Adapter PCR to Amplify from Plasma DNA

Linker mediated PCR was used to amplify DNA derived from the plasma of pregnant women. A standard protocol (Johnson, K. L., et al., Clin. Chem. 50:516-21 (2004)) was used to purify DNA from a 10 ml sample of anti-coagulated whole blood (maternal plasma). The samples were centrifuged twice to remove cells. The resulting plasma was passed over a DNA binding membrane. The DNA was removed from the membrane and the resulting DNA was digested with HpyCh4-IV (cuts at ACGT). Linkers were annealed and ligated, and PCR was performed using the top strand of the linker pair following a published protocol (Guillaud-Bataille, M., et al., Nucleic Acids. Res. 32:e112 (2004)). The linkers were slightly modified so that they created a MluI site when ligated to DNA digested with HpyCh4-IV. The linkers were as follows:

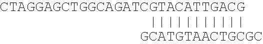

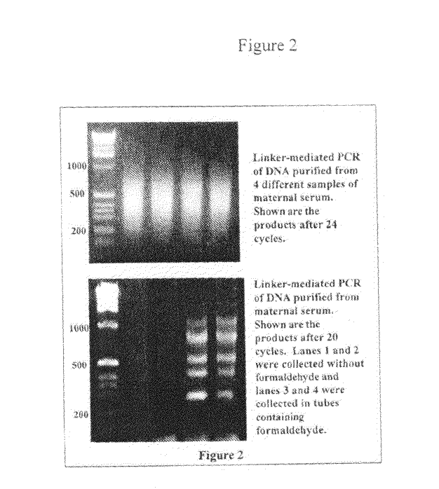

FIG. 2 shows representative examples of such amplifications and shows that PCR products are easily detected. To prove that the amplification was specific and linker mediated, PCR products were cloned using a standard TA cloning protocol. Ten random colonies were picked and sequenced, and in 9 of 10 cases, the sequence showed that the linker adapter was ligated to a bona-fide HypCH4-N site at each end. This experiment provides strong evidence that linker-adapter PCR can be used to amplify from plasma derived DNA.

Inspection of the bottom panel of FIG. 2 shows that linker-mediated PCR products generated with this protocol are strikingly different depending on whether one uses formaldehyde during the collection of maternal blood. Only two examples are shown, but this result was consistent among 12 separately collected samples. The laddering seen when blood is collected in the presence of formaldehyde is strongly reminiscent of apoptotic laddering, suggesting that formaldehyde favors the amplification of apoptotic fragments. Perhaps the fixation of proteins to DNA may result in DNA that is not digestible with restriction enzymes or the laddering may simply represent a dramatic reduction in complexity of the PCR product. According, it is preferred that maternal blood collections do not involve formaldehyde. This attitude is in keeping with a recent publication that refutes the notion that formaldehyde increases the proportion of fetal DNA. Chinnapapagari, S. K., et al., Clin. Chem. 51:652-5 (2005).

### Example 2

### Demonstration of Methylation Specific Amplification of Trophoblast/Fetal DNA

For the purpose of demonstrating differential methylation between trophoblast/fetal and whole-blood DNA, the highly reduced complexity resulting from the use of a rare cutting, AT rich enzyme is beneficial. Therefore, amplified representations from trophoblast/fetal and whole-blood DNA samples using AclI were prepared. Trophoblast/fetal DNA samples were derived from electively terminated first trimester pregnancies between 56 and 80 days gestation, and all whole-blood DNAs were prepared from normal adult volunteers.

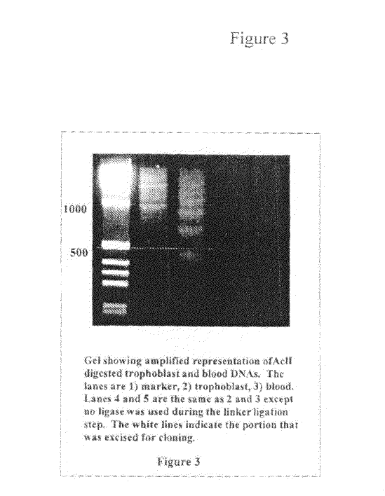

All amplifications were performed according to a published protocol. See Guillaud-Bataille, M., et al., Nucleic Acids. Res. 32:e112 (2004). Briefly, 0.5 ug of genomic DNA was digested with excess AclI in the recommended buffer. 25 ng of this was used to ligate to the linker/adapter pair. Following ligation, 2.5 ng of ligated DNA was used as template for PCR. After 14 cycles, 1/10^{th} volume of the product was used as template for a second round of PCR for 10 further cycles using the same primer. At this point, the products were displayed on a minigel (see FIG. 3). Consistent with the prediction of differential methylation, PCR products from trophoblast/fetal DNA consistently yielded more robust and differently appearing PCR products than did whole-blood DNA.

Fragments running between ˜500 and 1,000 bp were excised from the gel, digested with MluI to remove the linker/adapter and ligated to a MluI digested cloning vector. The linker/adapter was designed so that ligation to an AclI overhang results in the creation of a MluI site. These ligations were transformed into bacteria to yield mini-libraries of amplified AclI fragments from both trophoblast/fetal and whole-blood starting DNAs. At the point of cloning, trophoblast/fetal representations consistently yielded at least twice as many colonies, such that the best trophoblast/fetal mini-library contained about 8,000 recombinants in comparison with about 3,000 for the best whole-blood library.

Thirty-five random colonies from a trophoblast/fetal library were picked and their inserts sequenced. Analysis with the UCSC browser showed that all but four sequences corresponded to predicted AclI fragments less than 1 kb long, indicating that the digestion, linker ligation and amplification steps had all occurred as predicted. It should be noted that the cloning procedure strongly selects against unintended amplification products since the MluI site is only created when the linker is ligated to an AclI overhang.

When an attempt to clone PCR products utilized a TA cloning procedure, cloning efficiency was poor and a high percentage of clones reflected non-specific amplification products. Thus, it was concluded that a significant percentage of the DNA mass resulting from linker-mediated amplification is non-specific.

A total of 30 pairs of specific PCR primers were designed to amplify sub-segments of amplified AclI fragments. PCR was then performed using amplified AclI representations of whole-blood and trophoblast/fetal DNA as template. For these experiments, “second round” representations as described above were diluted 1 to 10 and were used as template for each of the specific primer sets, and amplifications were performed for 20 cycles under a standard set of conditions.

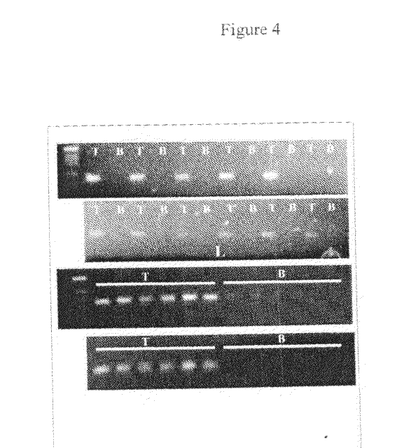

Primer sets that amplified from trophoblast/fetal but not from whole-blood representations, were further tested by amplifying from a set of 6 trophoblast/fetal and 6 whole-blood representations (see FIG. 4). Of these, 10 proved to be trophoblast/fetal “specific” in the sense that all 6 trophoblast/fetal representations yielded a clearly visible product while none of the 6 whole-blood representations yielded a product under the same conditions. This corresponds to a 10/30 or 33% chance that a randomly chosen amplified AclI restriction fragment is only present when the starting DNA was derived from trophoblast/fetal. This in turn, is reasonably consistent with the estimation (above) of the degree to which trophoblast/fetal DNA is globally hypomethylated.

Of the remaining 20 primer sets, 10 amplified equally from trophoblast/fetal and blood representations, and another 10 gave inconsistent results. When used to amplify from some AclI representations the expected products were amplified, while in other cases they were not. These results suggest that either: 1) there is extreme variation in methylation at relevant AclI sites; 2) that some AclI sites are altered by common SNPs; or 3) that PCR efficiency is affected by the presence of a SNP. Indeed, several examples in which SNPs altered AclI sites as well as examples of SNPs affecting PCR efficiency were identified. This type of sequence variation is expected and does not alter the conclusion that a high percentage of randomly chosen AclI amplicons are relatively trophoblast/fetal specific.

Of greater concern, was the observation that some primer sets amplified strong bands from trophoblast/fetal representations and much weaker bands from whole-blood representations. See the weak bands in the 3^{rd} panel from the top in FIG. 4. Further, as the number of PCR cycles was increased from 20 to 30, visible bands were amplifiable from whole-blood representations for almost all primer sets (not shown). Since the goal of the invention is the specific amplification of trophoblast/fetal DNA when it is mixed with other DNA, “leaky” amplification of non-trophoblast/fetal DNA is problematic.

In its narrow linear range, one can predict that 34 cycles of PCR corresponds to 10-fold amplification and that a 34 cycle difference in the threshold of detection corresponds to a log-fold difference in amount of template. To detect trophoblast/fetal DNA when it represents 1% of the DNA in a mixture, differential amplification of 6-8 PCR cycles would be necessary. Of the 10 trophoblast/fetal “specific” primer sets, only 1 or 2 fulfilled this stringent criterion.

Three possible causes for weak amplification from whole-blood representations were considered. First, small amounts of the starting genomic DNA still present in the amplified representation may provide enough template to get a weak product. It was calculated that of the 2.5 ng of starting genomic DNA, only a few picograms were present in the diluted representations, making it unlikely that this was the source of weakly amplifying bands after 20 cycles of PCR. However, this could potentially explain the amplification after 30 cycles. Second, methylation may be incomplete at many CpG sites. Sites that are highly, but not completely methylated would give rise to representations where the corresponding restriction fragment was present at a low but detectable level. Clearly, this explanation is likely to be valid at the extremes. Many sites might be methylated 99% of the time while others are methylated 99.9%. A third explanation for weak amplification from whole-blood amplicons is non-specific amplification during the formation of representations as described above. The process of denaturing, re-annealing and performing primer extensions with complex genomic DNA containing large amounts of repetitive sequence is certain to create large amounts of unintended products. To determine which of these three possibilities was the source of the “leaky” amplification from whole-blood DNA, an alternate scheme for representational amplification was devised.

### Example 3

### Isothermal Amplification of Circular Molecules Following Nuclease Digestion

To overcome the leaky amplification issues discussed in Example 2, the present inventors sought to develop a convenient amplification method that, like cloning, would strongly select for bona fide restriction fragments and against non-specifically amplified products.

To this end, genomic DNA was digested with AclI and linker ligations were prepared as described above. After 12 cycles of amplification with a linker primer, products were digested with MluI (which cleaves off the linker), stripped of low molecular weight (linker and primer) DNA by column purification, diluted to 0.5 ml in 1× ligation buffer (to create a very dilute solution) and treated with T4 DNA ligase overnight. The rationale behind this is as follows. The initial 12 cycles of PCR creates a size-selected representation of AclI fragments as well as unwanted, non-specific products. By digesting and ligating a very dilute solution, intramolecular self-ligation (circularization) of molecules with compatible sticky ends is strongly favored. The original starting DNA that has been melted and partially re-annealed 12 times is very inefficiently digested and circularized. The non-specifically amplified products that lack appropriate ends are also highly unlikely to form covalently closed circles.

After precipitation, the ligation mixture was treated by extensive digestion with nuclease Bal-31, an exonuclease that attacks the ends of single stranded and double stranded DNA: Circular molecules created by ligation are resistant to digestion, but extensive digestion will reduce linear molecules to single nucleotides. This is predicted to eliminate the starting genomic DNA as well as non-specifically amplified products. The nuclease treated ligations were then used as template for isothermal rolling circle amplification using a commercial kit (Amersham) following the manufacturer's recommendations. This results in an approximate ˜10,000 fold amplification that does not involve melting and reannealing. Dean, F. B., et al., Genome Res. 11: 1095-9 (2001). At the end of this procedure, the resulting DNA was diluted and used as template for PCR with the above described trophoblast/fetal “specific” primer sets.

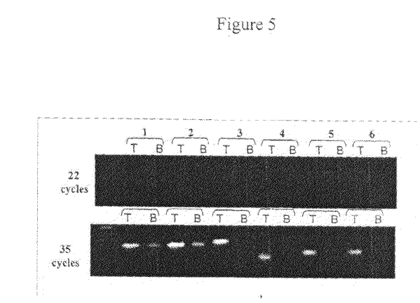

This analysis yielded a total of 5 (of the original 30) primer sets for which it was possible to clearly detect PCR products from trophoblast/fetal representations at 22 cycles while up to 35 cycles with whole-blood representations did not yield visible product. Examples of both success and failure are shown in FIG. 5. A 13 cycle difference in threshold of PCR detection corresponds to at difference in starting template of at least 1000 fold and predicts that these amplicons should be easily detected in DNA mixtures where the trophoblast/fetal component is only 1%.

The present inventors concluded that non-specific amplification in conventional linker-mediated amplification is a major source of “leakiness” or background and that the nuclease/isothermal amplification protocol improves this situation significantly. In addition, incomplete methylation is also likely to be present at many genomic sites, and this reduces the total number of strongly methylation specific amplicons.

### Example 4

### Amplification of Mixed Trophoblast/Fetal and Whole-Blood Samples

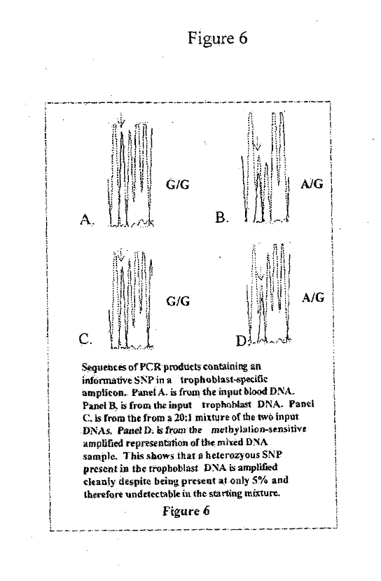

To test whether specific amplification of the trophoblast/fetal component of mixed DNA samples was possible, a trophoblast/fetal-specific AclI amplicon was identified that contains a common single nucleotide polymorphism (“SNP”) that alters a BanII site. The six trophoblast/fetal and six whole-blood test DNAs used above were genotyped for this SNP, and after finding a whole-blood/trophoblast/fetal pair with distinct genotypes at this locus, 10:1 and 20:1 mixtures of genomic DNA were prepared. The absolute amount of DNA in these mixtures was 25 ng, meaning that the trophoblast/fetal component in a 20:1 mixture was only ˜100 Pg and therefore less than the fetal component present in a 10 ml sample of plasma. Methylation-sensitive representations were prepared as described above, and diluted representations were then used as template for PCR. Products were analyzed by restriction digest as well as by direct sequencing (FIG. 6). Within the sensitivity of the assay, there was no evidence of amplification of the whole-blood component.

To further demonstrate the ability to selectively amplify the trophoblast/fetal component of DNA mixtures, the present inventors used simple sequence repeats (“SSR”) polymorphisms. Besides being much more informative than SNPs, with heterozygosities well over 50%, SSRs also offer the possibility of easily assessing the relative degree of amplification of alleles in the same DNA sample by measuring relative peak height or area with an automated sequencer.

To find AclI amplicons containing potentially polymorphic SSRs, plasmid DNA from a trophoblast/fetal mini-library (above) was digested with MluI and new linkers were ligated to the fragment ends. PCR using a primer consisting of (CA)₁₀ as well as a primer corresponding to the “bottom” strand of the linker was performed. This method was predicted to amplify portions of AclI fragments that contain CA repeats. PCR products were cloned and random colonies were picked and sequenced. Of 15 such sequences, all corresponded to predicted AclI fragments less than 1 Kb long, and five contained a CA repeat long enough to be potentially polymorphic. Specific primers flanking the CA repeat were synthesized and used for PCR on amplified representations and three of the five were shown to be trophoblast/fetal specific.

Ten of twelve test DNAs were shown to have heterozygous variations in CA length for one of these, and a pair of DNAs (trophoblast/fetal and whole-blood) with distinct genotypes was selected for making test mixtures consisting of 10:1 and 20:1 whole-blood and trophoblast/fetal DNA respectively.

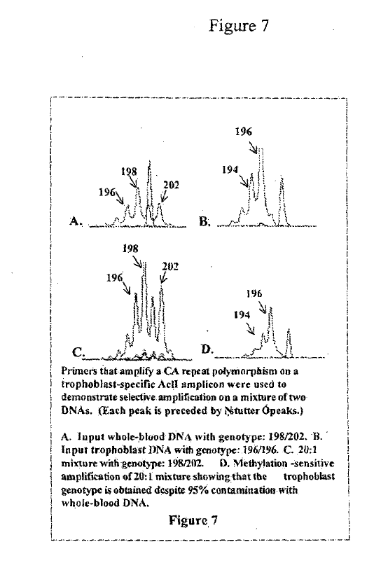

Mixed genomic DNA was then used to prepare methylation-sensitive representations, and dilutions of these were then used as template for PCR with primers for the polymorphism. The PCR products of each of the two starting DNAs as well as those amplified from the 20:1 mixtures are shown in FIG. 7. Clearly, amplification of this methylation-sensitive amplicon is at least 20 fold more efficient from trophoblast/fetal DNA that it is from whole-blood. The results of these experiments prove that with the methods of the present invention, preferential amplification of trophoblast/fetal DNA is possible.

### Example 5

### Microarray Analysis for Large-Scale Identification of Trophoblast/Fetal-Specific Amplicons

Development of a library of trophoblast/fetal-specific amplicons is a first step towards aneuploidy testing as described below.

Comparative hybridization to custom-made oligonucleotide microarrays is now a routine, commercially available technology that has been extensively used to assess genomic copy number differences. The same technology provides an ideal method for the large-scale identification of trophoblast/fetal specific amplicons. To achieve this goal, methylation-sensitive representations prepared separately from trophoblast/fetal and whole-blood DNA are labeled with different fluorochromes and comparatively hybridized to arrays of oligonucleotides that correspond to predicted restriction fragments for a given methylation-sensitive enzyme. As opposed to array hybridization for copy number changes, where differences in hybridization level are extremely subtle, those oligonucleotides (array addresses) that hybridize exclusively to the trophoblast/fetal probe are identified, reflecting 0 or near 0 digestion of corresponding restriction sites in DNA derived from blood. By performing such microarray analyses using probes made from multiple different trophoblast/fetal samples, those amplicons that consistently show highly differential amplification are identified and used to provide a catalogue of a large number of trophoblast/fetal-specific amplicons located on target chromosomes.

### Choice of Restriction Enzyme

In the studies described above, where the goal was to demonstrate the existence of trophoblast/fetal-specific amplicons, a rare cutting enzyme that resulted in amplified representations with extremely reduced complexity was deliberately employed. For the purpose of future prenatal diagnosis, several hundred trophoblast/fetal specific amplicons per chromosome for the target chromosomes, 13, 18 21 X and Y are obtained, and, because of the low average molecular weight of plasma derived DNA, the focus is on short segments. Clearly, enzymes such as AclI result in too few fragments for this purpose, and therefore, a more frequently cutting enzyme for microarray analysis is used. The enzyme HpyCh4-IV is ideal for producing representations for microarray experiments. This enzyme is the only commercially available enzyme whose recognition sequence (which is ACGT) fulfills the criterion of having either A or T at positions other than the central CpG. In a genome with balanced proportions of A, C, G and T, there should be 16 fold more sites for HpyCh4-IV than for AclI, and this, in turn, would predict 2400 fragments between 100 and 1500 bp long for chromosome number 21. In fact, the true number of HpyCh4-IV fragments of size 100-1500 predicted for chromosome 21 is 17,152, reflecting the extremely uneven distribution of CpG dinucleotides with respect to AT rich sequence. If one makes the assumption that 80% of sites are blocked by methylation in trophoblast/fetal DNA, one can guesstimate that the true number of chromosome 21 fragments in the target size range is 2-3000. If 15% are trophoblast/fetal specific, then 300-450 such amplicons are predicted.

### Array Construction

Current technology allows the production of arrays containing ˜380,000 different oligos, enough to allow the assessment of over half of all HpyCh4-IV fragments in the entire genome in a single experiment. However, to perform this type of analysis on 10 sample pairs would require a minimum of 20 such arrays and would therefore be excessively expensive. As a cost saving alternative, an array format in which 4 identical arrays is provided, each consisting of ˜98,000 oligos each, are synthesized on the same “chip”. A single “chip” of this type allows 4 hybridizations, which is sufficient for 2, color-reversed, duplicate hybridizations. 98,000 oligos provides sufficient space to query ˜12,000 fragments on each of the 4 relevant chromosomes (13, 18, 21 and X) with each oligo in duplicate. 12,000 is sufficient to represent the majority of 100-1500 bp fragments located on chromosome 21, and this, in turn, is expected to yield several hundred trophoblast/fetal-specific amplicons per chromosome. Because all Y segments are fetal-specific, only 1000 Y segments are represented in the arrays. This is predicted to yield ˜200 Y chromosome amplicons, which should be more than sufficient.

### Oligonucleotides

A database containing the sequence of all ˜17,000 predicted HpyCh4-IV fragments on the 21, 18, 13, X and Y chromosome between 100 and 1,500 bp in length are prepared. These files are then used for probe design and array synthesis. Because of the low molecular weight of plasma DNA, the maximum possible number of short fragments will be represented in arrays. Since about 50% of fragments less than 400 bp will not have suitable sequence for oligonucleotide design, this will leave about 2,500 to be represented in the array. All arrays also contain a series of negative control oligonucleotides.

### Trophoblast/Fetal Samples

As discussed above, first trimester trophoblast/fetal DNA is used because of two considerations: 1) the differences in methylation between trophoblast/fetal DNA and other DNA are more pronounced in early gestation; and 2) a first trimester diagnostic method is desirable. Using the same logic, microarray hybridizations using representations amplified from trophoblast/fetal derived from pregnancies of 56-84 days are performed. These samples may be collected from electively terminated pregnancies, and DNA will be prepared by routine proteinase-K digestion followed by phenol/chloroform extraction.

### Non-Trophoblast/Fetal Samples

10 randomly chosen female samples are pooled rather than attempting to choose appropriate individual whole-blood DNA samples. By pooling blood derived DNA, a single representation with an average methylation profile is produced.

It is assumed that maternal DNA contaminating samples obtained from the cervix is derived from the cervical epithelium and is thus similar to DNA derived from skin fibroblasts. There have been no systematic studies comparing the methylation in blood and skin derived DNA, but there is no reason to believe they are different in this regard. In the past, gene mapping experiments were performed in which Southern blots with methylation sensitive digests were hybridized to more than 20 different probes and no differences between blood and fibroblast DNA were ever seen.

### Probe Synthesis

The nuclease/rolling-circle amplification protocol described above is used to prepare methylation-sensitive representations of trophoblast/fetal and non trophoblast/fetal DNAs. 0.5 ug of each genomic DNA is digested with excess HpyCh4-IV. 25 ng of this digest is ligated to the linker pair and 1/10th of the ligation is used to perform PCR for 12 cycles. In the above examples using AclI digests, legitimate ligation of the linker to the fragment end produced a MluI site (ACGCGT) and the same result is obtained when using HpyCh4-IV which cleaves after the A to leave CGT. After 12 cycles of PCR, the resulting products are digested with MluI and circularized as above. Following ligation, remaining linear DNA is digested with nuclease Bal-31, and after buffer exchange with a Sephadex G50 column, isothermal rolling-circle amplification is performed using a commercially available kit (Amersham Bioscience). At this point, the DNA is checked on a minigel to determine whether appropriate products are present. The DNA yield using this protocol is routinely between 3 and 5 ug, but because only a portion of the circularized PCR product is used for amplification, it can easily be scaled-up for larger quantities. After determining quantity by fluorometry and quality by running MluI digested products on a minigel, DNA is supplied to an array manufacturer, such as NimbleGen, for probe labeling and array hybridization.

### Interpretation of Microarray Data

Processing of raw data is an important first step. For each array address the signal intensity (with respect to control oligos) is assessed. Spots that prove unreliable are excluded from analysis. For each array address with an adequate signal, the ratio of intensity of the two signals (Cy3 and Cy5) is determined. Because log transformed ratios have better statistical properties than simple ratios, all will be log(base 2) transformed. Array data is normalized by subtracting the median log₂ ratio for an entire array from each individual value of the array. Since each oligo is present in duplicate, the normalized ratios of duplicate addresses are averaged, and these means are averaged with the corresponding color-reversed mean ratio of the same duplicate address. Thus, the final value for each segment is based on four hybridizations and their corresponding log₂ mean ratios. This analysis is easily accomplished with existing software packages.

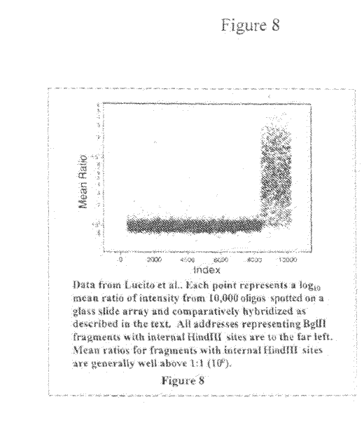

Locating those amplicons that are present in trophoblast/fetal representations but are absent or nearly absent in whole-blood representations is quite different than in the typical genomic comparison experiments where one is looking for subtle differences in hybridization ratios in genomically contiguous array addresses. Data from Lucito et al., Genome Res. 13; 2291-305 (2003) provides an example of how the data will likely appear. See FIG. 8. These authors performed comparative hybridizations to glass slide arrays of 10,000 oligonucleotides that corresponded to BglII fragments. One hybridization probe consisted of a “complete” BglII representation of genomic DNA and the other consisted of a similar representation except that the DNA was also digested with HindIII, largely eliminating all fragments with an internal HindIII site. This creates a situation similar to the present invention, where one representation contains elements that are essentially missing from the other representation. As is evident the figure, the log₁₀ mean-ratio signals vary from 0 to well over 1, reflecting a >10 fold difference in intensity for many segments. The results for the present invention arrays will be similar to these, but, because the probe amplification method creates much less non-specific amplification than that used by these authors, it is likely that a higher percentage of addresses with log₁₀ mean ratios greater than 1 will be seen.

Those addresses with a 10 fold or greater mean-ratio are considered to be “trophoblast/fetal-specific.” Clearly, those addresses with the highest mean ratios are the most desirable. The analysis of each hybridization yields a list of probe addresses with signals that meet this criterion, and a pair-wise comparison of the 10 planned hybridizations yields a final list of those addresses that are consistent among the samples, providing the desired catalogue of trophoblast/fetal specific HpyCh4-IV amplicons for the five relevant chromosomes.

### Example 6

### Amplification of Fetal-Specific Polymorphisms from Maternal Plasma and Cervical DNA

The amplification of fetal polymorphisms is also a possible avenue for non-invasive aneuploidy testing. QF-PCR of STR polymorphisms has been shown to be highly successful for the rapid diagnosis of aneuploidy in conventional prenatal diagnosis (Nicolini et al., Hum. Reprod. Update 10:541-48 (2004)) and can adapted to for use with methylation-sensitive-representation. Therefore, useful polymorphisms located on the methylation specific amplicons defined in Example 5 are identified, and fetal alleles of these polymorphisms in cervical and plasma DNA samples are detected.

### Discovery of Useful Polymorphisms

For the purposes of genetic mapping, SNPs have become the most useful and most plentiful type of polymorphism. Although millions of SNPs are in public domain databases and assay methods for SNPs abound, their use for the detection of aneuploidy presents a greater challenge than STR polymorphisms. Not only are they less polymorphic, but methods to use them for aneuploidy testing (Pont-Kingdon, G. et al., Clin. Chem 49: 1087-94 (2003)) depend on equal amplification of alleles that may not be realistic in the context of methylation-sensitive-representations. With this in mind, useful STRs located on methylation specific amplicons are identified.

To demonstrate the feasibility of locating STRs located on methylation specific amplicons, chromosome 21 was searched for HpyCh4-IV fragments that contain potentially polymorphic runs of simple sequence Of the ˜17,000 predicted fragments, nearly 400 contain STRs that are likely to be polymorphic. See Table 1.

TABLE 1
Potential Chromosome 21 Polymorphisms

| | 100-400 bp | 400-1,500 bp | total |
|---|---|---|---|
| CA/TG (10 or >) | 47 | 260 | 307 |
| Tri or tetra (10 | 10 | 58 | 68 |
| or >) | | | |

The arrays described in Example 5 above contain oligos corresponding to as many of these as possible, thus increasing the chances that potentially polymorphic sites will be found on methylation specific amplicons. Given that about 15% of fragments are likely to be highly methylation specific, up to 60 potentially polymorphic trophoblast/fetal-specific amplicons on chromosome 21 are identified. Because they generally yield more easily interpreted PCR products, tri and tetra nucleotide repeats are used. A primer pair flanking the target polymorphism is designed. One of the two primers is labeled with a fluorochrome for easy fragment length analysis on an automated sequencer, and PCR is performed on 10 random genomic DNA samples. Markers with a reasonable heterozygosity are revealed in this way, and promising candidates are further tested.

### Tests of Amplification on Mixed DNA Samples

Existing trophoblast/fetal and whole-blood DNA samples are genotyped with respect to polymorphisms identified above, and mixed sample pairs with distinct genotypes are prepared. Data indicates that detection of the trophoblast/fetal genotypes on mixtures where the trophoblast/fetal component is 5% of the total starting DNA is feasible, so we 20:1 mixtures are first tested, followed by test analysis with 50:1 and 100:1 ratios.

### Testing for Fetal-Specific Amplification

Identified polymorphisms that function well in the above tests, are used to test whether fetal alleles can be amplified from maternal samples. For this purpose, samples of both maternal and fetal DNA re obtained for each maternal plasma and/or cervical lavage sample. For plasma samples from ongoing pregnancies, fetal DNA is obtained from the CVS specimen and for lavage samples, it is obtained from the termination specimen. For those cases where there is a maternal blood sample but not direct fetal sample, the availability of a paternal sample will allow identification of fetal-specific alleles. Maternal and fetal (or paternal) samples are genotyped with respect to these polymorphisms using fluorescent PCR. With 5-10 loci in hand, it is likely that one or two loci will be informative for almost all pregnancies. Samples that are predicted to allow the unequivocal identification of fetal alleles are selected.

As suitable samples are identified, methylation-sensitive representations of the mixed fetal/maternal samples (either cervical or plasma) are prepared as described above. Because size selection of plasma DNA appears to significantly enrich for fetal DNA (Li et al. Clin. Chem. 50:1002-11 (2004)), size selection is as follows. After the initial digestion, linker ligation and 12 cycle amplification, the PCR products are loaded on a 2% agarose minigel. A gel slice containing fragments between 100 and 400 is excised and used for the subsequent step of digestion with MluI, circularization, and isothermal amplification. This protocol achieves the same advantages as size-selecting the DNA directly. For cervical lavage samples (obtained according to the protocol above) from the entire cell pellet obtained from the lavage specimen is prepared, and used for methylation-sensitive amplification.

The amplified products are used as template for amplification of informative polymorphisms, and fragment analysis reveals whether fetal-specific alleles can be amplified.

### Testing for Aneuploidy

Samples from pregnancies with a high suspicion of trisomy 21 are obtained as they become available. Methylation-sensitive amplified representations are prepared as described above from these samples. The same procedure for size selection as discussed above is used. After determining the true fetal genotype with respect to the panel of trophoblast/fetal specific chromosome 21 markers (using DNA from the CVS or termination), the same set of PCRs on the amplified representations are run.

### Example 7

### Use of Methylation-Specific-Representation for Microarray Comparative Genome Hybridization (“CGH”)

### General Considerations

Comparative hybridization of oligonucleotide arrays is capable of detecting tiny genomic deletions and duplications (Sebat et al. 2004; Jobanputra et al. 2005; Selzer et al. 2005). The detection of cytogenetically visible deletions and whole chromosome aneuploidy is comparatively simple with this technique. Historically, a key factor in the success of this technique is the fact that amplified representations reduce the complexity of the probe, making the proportion that is actually homologous to the target oligonucleotides much larger. More recently, improvements in techniques for probe labeling have made it possible to use directly labeled, whole genomic probes on oligonucleotide microarrays. Several groups have reported the use of this technique to detect small, single copy number changes, proving that highly complex probes are routinely successful (Brennan et al. 2004; Selzer et al. 2005; Hinds et al. 2006). Thus, comparative hybridization of methylation-specific representations to arrays of oligonucleotides that correspond to trophoblast/fetal-specific amplicons may be used to detect fetal aneuploidy.

In this scheme, methylation-sensitive representations are prepared from DNA samples from plasma or cervical samples of pregnant women as described above. The amplified representations from two different individuals, one a normal control and the other of unknown karyotype, are then used as comparative hybridization probes to the set of trophoblast/fetal-specific oligonucleotide targets defined in Example 5. If the two pregnancies both have normal karyotypes (and are the same sex), then similarly balanced hybridization signals are expected for all 5 chromosomes represented in the array. If one of the two pregnancies has a whole chromosome aneuploidy (e.g. trisomy 21), then the oligo set representing that chromosome would be expected to show an overall relative imbalance of signal of the two fluorochromes when compared to the other 4 chromosomes. Fetal sex would be reflected by the mean ratios of signals from the sex chromosome probe sets. The degree of signal imbalance for any given address in the array need not be large since the data from all ˜100 addresses representing that chromosome are considered in aggregate.

The main parameter determining the success of this scheme is the degree to which trophoblast/fetal-specific amplicons are represented in the hybridization probe, Clearly, if one started with pure fetal DNA, the detection of aneuploidy with this technique would be trivial. Likewise, if one started with an equal mix of fetal and maternal DNA, the methylation-sensitive representation of trophoblast/fetal sequence would be over well over 50% of the probe mass and aneuploidy detection would be expected to work just as well as if one started with pure fetal DNA. The ease with which this scheme is successful clearly lessens as the proportion of trophoblast/fetal DNA diminishes. With this in mind, a situation where the starting DNA is 1% from the trophoblast/fetal and 99% maternal is considered and discussed below.

Methylation-sensitive amplification on such a 99:1 mixture will have 2 major consequences. First, the overall sequence complexity will be reduced by ˜98% (see below); and second, trophoblast/fetal-specific fragments will be present. In terms of DNA mass, the trophoblast/fetal-derived component (both specific and non-specific) will still be only about 1.5-2% of the total. To the extent that trophoblast/fetal DNA is hypomethylated, its efficiency of amplification and proportion is increased, but this effect is small, since data indicates that no more than 30% of fragments in trophoblast/fetal representation are trophoblast/fetal-specific. Can a hybridization probe that represents only ˜2% of the total mass of the DNA in the probe mixture provide a reliable signal? This depends on the overall complexity of the probe mix. To calculate the approximate complexity of the probes that are produced by our methylation-sensitive procedure, it was considered that chromosome 21, which consists of ˜49 Mb of DNA, contains ˜17,000 HpyCh4-IV fragments of size 100-1500 bp. Methylation will block the amplification of 80-90% of these making a total number of amplified fragments ˜1,700-3,400. Since the mean size of these is ˜500 bp, the total amplified complexity is about 0.85-1.7×106 or about 1.5-3% of the total starting sequence. This corresponds to an approximate 97-98% reduction in complexity compared to genomic DNA. A hybridization probe produced from a DNA sample that contained 1% trophoblast/fetal DNA would be expected to have only ˜2% of its mass derived from the trophoblast/fetal component, but, because the overall complexity is reduced by 98%, the effective concentration of trophoblast/fetal specific probe is similar to the concentration of any given segment in a whole-genome probe. Therefore, it is predicted that probes derived from methylation-specific amplified representations of DNA that was at least 1% derived from trophoblast/fetal should provide hybridization signals equal to or better than those from whole-genome probes. Obviously, higher starting proportions of trophoblast/fetal DNA would provide proportionally stronger signals.

### Experimental Design

### Arrays

Example 5 discusses oligonucleotide probes for trophoblast/fetal specific amplicons from the 5 relevant chromosomes. As stated above, it is estimated that the number of such amplicons will be about 200 per chromosome or about 1,000 in total. Any array could be used. For example, an array where conventionally synthesized oligos are immobilized on glass slides may be used. A number of procedures for producing oligonucleotide arrays on glass slides have been described. (Guo et al. Nucleic Acids Res 22:5456-65 (1994); Zammatteo et al. Anal Biochem 280:143-50 (2000); Kimura et al. Nucleic Acids Res 32:e68 (2004)). The oligonucleotide sequences corresponding to the ˜1,000 anticipated trophoblast/fetal-specific as determined in Example 5, and conventionally synthesized oligos for ˜500 of these (˜100 per chromosome) are obtained. Arrays of these oligos are produced following existing protocols. All oligos are spotted in duplicate, and non-homologous oligos with similar predicted Tm are spotted as negative controls. As positive hybridization controls, ˜10 amplicons per chromosome that consistently hybridized equally to trophoblast/fetal and peripheral blood derived probes (from Example 5) are also included. Those oligos that do not function well in the test hybridizations described below are removed from the array and replaced with others that may function better.

### Hybridization Probes for Assessment of Arrays

Initially it is determined whether reliable trophoblast/fetal-specific hybridization signals can be obtained through comparative hybridization as envisioned above. Initial attempts at hybridization with these arrays utilize “artificial” mixtures of cytogenetically normal trophoblast/fetal and whole-blood DNA rather than actual maternal samples. Initially, 3 such mixtures (A, B and C) are prepared from 3 separate trophoblast/fetal/blood DNA pairs, and in all 3, a 1:1 ratio of the 2 DNAs will be used. In each of the 3, the whole-blood DNA is from a female and while two of the trophoblast/fetal samples are male and the third is female. Methylation-specific representations are then prepared following the same protocol as above. Following linearization and determination of average size and concentration, probe labeling will follow existing protocols. (Ushijima et al. Proc Natl Acad Sci USA 94:2284-9 (1997); Brennan et al. Cancer Res 64:4744-8 (2004)). The key to these is the use of directly labeled random primers as well as labeled dNTPs during Klenow extension. All three mixtures (A, B and C) will be labeled with both Cy3 and Cy5 in separate reactions (6 total probes) so that each mixture can be comparatively hybridized to itself as well as to the others.

Probes made from 1:1 mixtures should result in very intense hybridization signals and as the proportion of trophoblast/fetal DNA decreases, the signal intensity will decrease correspondingly. Performing comparative hybridizations of the 6 possible pairs that arise from 3 trophoblast/fetal/whole-blood mixtures (AA, AB, AC, BB, BC and CC), important data on the reliability and reproducibility of the technique is produced.

### Data Analysis

As in example 5, raw array data is assessed for quality by determining signal strength with respect to positive and negative hybridization controls as well as replicate consistency, and unreliable spots are excluded. For each hybridization, the mean signal ratio associated with each array address is determined. Ratios are log₂ transformed and normalized by subtracting the median log ratio value of the entire array from each individual value of the array. Each mean-ratio is based on four hybridizations since each oligo is spotted in duplicate and each hybridization is performed twice, with color reversal. Normalized log ratios for the 3 autosomes are centered around 0 for all these comparisons since they are all cytogenetically normal. Standard analysis of variance techniques (“ANOVA”) is applied to obtain a preliminary estimate of the degree of variation seen between hybridizations performed with cytogenetically normal samples. The ratio of signal from the sex chromosomes should be obvious from this analysis. All three possibilities of male to male, female to male and female to female are tested. As in all such situations, when XX is comparatively hybridized to XY, there should be a ˜2:1 X derived signal in one color and a very obviously discrepant ratio of Y signal in the other color.

After a 1:1 ratio of trophoblast/fetal and whole-blood DNA results in reliable hybridizations, the same set of control hybridizations using the same DNAs, but with 10:1 ratio of whole-blood:trophoblast/fetal is performed. Similarly, the entire exercise using 50:1 mixtures is performed. This exercise is important for determining the initial proportion of fetal DNA a sample must contain to be successfully analyzed by this technique. If a 50:1 ratio of whole-blood to trophoblast/fetal DNA is usable in this context, then it should be possible to use maternal plasma and/or cervical samples as the starting material for amplified representations.

### Aneuploidy Detection

The data derived from the above experiments allow the detection of aneuploidy using methylation-specific amplification. To this end, “artificial” mixtures of normal whole blood DNA and trisomy 21 trophoblast/fetal DNA at ratios of 10:1 and 50:1 are prepared. These mixtures are used to make methylation specific hybridization probes with both Cy3 and Cy5 and these are comparatively hybridized to themselves as well as to the 3 cytogenetically normal mixtures described above. In a comparative hybridization of a trisomy 21 mixture with itself, the chromosome 21 mean ratio is similar to the mean ratios for the other 2 autosomes since each probe has 3 copies of chromosome 21. When a trisomy 21 mixture is comparatively hybridized to a normal mixture, the chromosome 21 mean ratio is significantly different from the other autosomes, reflecting three copies of chromosome 21 in one sample and 2 in the other.

To formalize this analysis, the mean log₂ ratios across all 3 autosomes is compared using ANOVA. This provides the ability to test the global null hypothesis that the mean log ratios of all chromosomes are the same, and rejecting the null hypothesis would imply that at least one chromosome has an imbalance. By performing pair-wise comparisons between log₂ mean ratios of data from individual chromosomes to that from the other chromosomes it is possible to determine which chromosome is providing an unbalanced signal. Because 3 additional hypotheses will be tested, a Bonferroni correction is applied. Thus, the global null hypothesis will be tested at a 0.05 level and, if rejected, the chromosome-specific pair-wise hypotheses would be tested at the 0.015 level.

With approximately 100 array addresses per chromosome, there is a very high power to detect aneuploidy. Theoretically, it is desired to detect any increase in copy number that corresponds to a mean ratio of 1.5 (0.58 on the log₂ scale). Experience however has shown that increases in copy number of 3:2 (as in trisomy) can correspond to observed ratios as low as 1.15 (0.2 on the log₂ scale) due to noise in the data. Power analysis indicates that for a given pair-wise comparison there will be a greater than ˜99.99% power to detect an increase in copy number of 0.2 (log₂ scale) if one assumes that the standard deviation of log₂ mean signal ratios ranges from 0.1 to 0.2 (typical values) and assuming a type 1 error rate of 0.01. Even with a standard deviation of 0.4, which would represent a very noisy and poor quality hybridization, the power to detect aneuploidy is 97%.

### Testing of Real Maternal Samples

The data from all of the above described hybridizations comparing known normal and known trisomy DNA mixtures allow for the determination of the proportion of fetal DNA that is necessary for obtaining reliable hybridizations and hence determination of trisomy. For all the reasons cited above, it is believed that even low proportions of fetal DNA (2-5%) will be successful. Accordingly, as in example 6, analysis with both plasma and cervical lavage derived DNA is performed, since each type of sample has its strengths and weaknesses. Sample collection from ongoing pregnancies (maternal blood samples) as well as from elective termination cases is performed as in example 6. Likewise, the preparation of amplified representations is identical as well. In fact, the same amplified representations can be used for both example 6 as well in this example. Comparative hybridization is performed with pairs of samples with normal fetal karyotypes as determined by routine cytogenetic analysis or by QF-PCR.

As a practical matter, four such pregnant samples as well as two non-pregnant controls in both the plasma DNA group as well as the cervical lavage group are used, giving a total of 12 samples. Pair-wise comparison results in 15 analyses for each group. The actual number of hybridizations is 30 since each is performed with dye reversal. Analysis of the data from these hybridizations is performed as described above, and this provides several pieces of critical information: the ability to reliably obtain signals that are above background; the range of normal variation that is expected in signal intensity; and whether comparisons of log mean ratios between chromosomes appropriately centered around 0 for cytogenetically normal samples.

Reliable fetal hybridization signals can be obtained from methylation-sensitive amplified representations of maternal samples and can then be used to detect aneuploidy. As discussed above, this effort begins with trisomy 21 since this is the most relevant and the most available. Probes are prepared from cervical and plasma samples obtained from patients who have had trisomy 21 pregnancies documented. These are comparatively hybridized with each other as well as to probes prepared from normal cases. Statistical analysis proceeds exactly as in the experiments described above. Because reliable hybridization is obtained, the power to detect aneuploidy is extremely high.

In addition to the prenatal diagnostic possibilities, the methods of the present invention can be used to further understand biology. For example, the microarray analysis of the type discussed in example 5 can be used to examine the gestational age dependence of trophoblast/fetal methylation. Preliminary observations suggest that there are broad changes in methylation as pregnancy progresses, but there is no understanding of what role such changes may have in placental gene expression or function. Likewise, there have been no investigations into the role of placental methylation in disease. Beyond this utilitarian goal envisioned in this application, the development of a method for the comprehensive assessment of methylation differences between trophoblast/fetal and somatic DNA derived from other sources is likely to have many interesting biologic applications. For instance, one could look for global alterations in trophoblast/fetal methylation in disease states such as preclampsia, intrauterine fetal growth restriction, molar pregnancy and others. In the long-run, the combination of methylation-sensitive amplification and microarray hybridization will allow the systematic evaluation of placental methylation in disease states such as early pregnancy failure, intrauterine growth restriction, preclampsia and others.

1. A method for selective amplification of fetal DNA from a mixed fetal and maternal DNA sample comprising
a) isolating DNA from a mixed fetal/maternal DNA sample;
b) digesting the DNA with a methylation specific enzyme;
c) ligating the digested DNA with a linker
d) subjecting the digested DNA to linker-mediated PCR amplification to obtain amplified PCR products;
e) removing linker and primer DNA from the amplification products;
f) circularizing the amplified PCR products;
g) subjecting the circularized PCR products to exonuclease digestion to reduce any uncircularized DNA to single nucleotides; and
h) subjecting the products from step g to isothermal rolling circle amplification to selectively amplify fetal DNA to produce methylation-sensitive representations from fetal DNA.

2. The method of claim 1 wherein the methylation specific enzyme is HpyChIV-4, ClaI, AclI or BstBI.

3. The method of claim 1 wherein the linker mediated PCR amplification is performed for 12 cycles.

4. The method of claim 1 wherein exonuclease digestion is With Bal-31.

5. A method of identifying a fetal-specific amplicon comprising,
a) separately preparing methylation-sensitive representations from fetal DNA and whole-blood DNA using the method of claim 1;
b) labeling the fetal DNA and the whole blood-DNA to produce labeled fetal DNA probes and labeled whole-blood DNA probes;
c) hybridize labeled DNA probes to two identical arrays of oligonucleotides, wherein said arrays of nucleotides correspond to predicted restriction fragments for a given methylation-sensitive enzyme;
d) compare the two arrays with each other to locate an oligonucleotide that hybridizes exclusively to a fetal DNA probe;
e) identifying the hybridized oligonucleotide from step d as a fetal-specific amplicon.

6. The method of claim 5 wherein the fetal DNA probe and the whole-blood DNA probe are labeled with two different labels and wherein the hybridization of labeled probes is to one array.

7. The method of claim 5 wherein the methylation sensitive enzyme used in step a is HpyCh4-IV.

8. The method of claim 5 wherein the fetal DNA is obtained from first trimester pregnancies

9. The method of claim 8 wherein the fetal DNA is obtained from pregnancies of about 56-84 days.

10. A library of fetal-specific amplicons produced by the method of claim 5.

11. An array comprising the library of the fetal-specific amplicons of claim 10.

12. A method for determining whether the copy number for a predetermined locus of fetal DNA in a mixture of fetal and maternal DNA is either reduced or increased as compared to a normal copy number at the predetermined locus, comprising
a) selectively amplifying the predetermined locus of fetal DNA in the test sample and in a control sample using the method of claim 1, wherein said control sample has a normal copy number at the predetermined locus of fetal DNA;
b) comparing the amount of the amplified DNA in the test sample to the amount of amplified DNA in the control sample; and
c) correlating the reduced amount of amplified DNA to a reduced copy number or an increased amount of amplified DNA to an increase in copy number.

13. The method of claim 12, wherein the comparison includes normalization of the amplified DNA from the predetermined locus to DNA amplified from a control locus present at the same copy number in the test sample and the control sample.

14. A method for determining in a test sample whether a copy number for a predetermined locus is either reduced or increased as compared to a normal copy number, comprising
a) selectively amplifying fetal DNA in the test sample and in a control sample using the method of claim 1, wherein said control sample has a normal copy number at the predetermined locus;
b) labeling DNA from the test sample and the DNA from the control sample from step a with a label to produce labeled test DNA probes and labeled control DNA probes;
c) hybridize labeled test DNA and labeled control DNA probes to an array of fetal-specific amplicons of claim 11;
d) compare the amount of hybridization between the test DNA probes and the control DNA probes to determine signal strength;
e) correlate the signal strength with either an increase or decrease in copy number at the predetermined locus in the test sample.

15. The method of claim 14 wherein the test sample DNA and the control sample DNA are labeled with two different probes and wherein the hybridization is to one array.
